(19) United States
(12) Patent Application Publication
(10) Pub. No.: US 2012/0282314 A1
(43) Pub. Date: Nov. 8, 2012

(54) DERMATOLOGICAL/COSMETIC SKIN DEPIGMENTING COMPOSITIONS

(75) Inventors: Isabelle PELISSON, Vallauris (FR); Itaru SUZIKI, Fayence (FR)

(73) Assignee: GALDERMA RESEARCH & DEVELOPMENT, Biot (FR)

(21) Appl. No.: 13/551,437

(22) Filed: Jul. 17, 2012

Related U.S. Application Data
(62) Division of application No. 12/588,972, filed on Nov. 4, 2009.
Application No. 12/588,972 is a continuation of application No. PCT/FR2008/050731, filed on Apr. 22, 2008.

(30) Foreign Application Priority Data
May 4, 2007 (FR) 0754886

(51) Int. Cl. A61K 8/34; A61K 8/02; A61Q 19/08; A61Q 19/00; B82Y 5/00

(52) U.S. Cl. 424/401; 424/62; 977/773; 977/915

(57) ABSTRACT

Depigmenting compositions having improved effectiveness in the dermatological or cosmetic treatment of pigmentation contain, in a physiologically acceptable medium, at least one compound selected from among rucinol and salts thereof, and at least one retinoid; such compositions are also useful in the prevention and/or dermatological treatment of skin hyperpigmentation, and also for the cosmetic treatment of photoinduced or chronological aging of the skin or of the skin appendages.

### CROSS-REFERENCE TO PRIORITY/PCT APPLICATIONS

This application is a divisional application of copending Unites States patent application Ser. No. 12/588,972, filed Nov. 4, 2009, which is a continuation of PCT/FR 2008/050731, filed Apr. 22, 2008 and designating the United States (published in the French language on Dec. 11, 2008 as WO 2008/148968 A1; the title and abstract were published in English), which claims priority under 35 U.S.C. §119 of application Ser. No. 07/54886, filed in France on May 4, 2007, each earlier application hereby expressly incorporated by reference in its entirety and each assigned to the assignee hereof.

### BACKGROUND OF THE INVENTION

1. Technical Field of the Invention

The present invention relates to depigmenting compositions, to methods for preparing such compositions, and to various applications thereof.

2. Description of Background and/or Related and/or Prior Art

Skin hyperpigmentation is a common disorder which manifests itself through the appearance of brown or colored spots on the skin due to the accumulation of melanin, and which provides the skin with a heterogeneity. The pigmented spots may appear on all parts of the body, in particular on the back of the hands, on the face, the neckline and the head of men.

Several factors can contribute to the development of hyperpigmented lesions, the most common being familial predisposition, hormones, exposure to sunlight, and skin aging. In addition, pigmented spots can appear following attacks on the skin or skin inflammations. An increase in melanin production may thus be caused by a cutaneous inflammatory process, for example after traumas, eczematous eruptions, or other skin irritations.

Among the depigmentation disorders, age spots or solar lentigines are a common form of hyperpigmentation. These are due to damage caused by sunlight, and usually appear on the back of the hands and the arms, on the neckline or else on the face. These spots are darker than freckles or ephelides, and persist in winter.

Consequently, there exists a real need for an effective risk-free treatment for the symptoms of photoaging, in particular the hyperpigmentary spots induced by exposure to ultraviolet rays.

Melasma or chloasma lesions are more widespread than age spots and located on the face. They are most commonly due to hormonal changes. Pregnancy, for example, can trigger the overproduction of melanin which causes the “pregnancy mask”.

Changes in skin color can result from external causes, for example skin diseases such as acne, or skin lesions. Freckles are also small brown spots which can appear anywhere on the body, but are most common on the face and the arms. Freckles are an inherited characteristic.

Postinflammatory hyperpigmentation (PIHP) is also a common pigmentation disorder which may be the consequence of various skin disorders just as it may be the consequence of therapeutic procedures. This excess skin coloration may be subsequent to infections, allergic reactions, traumas such as an abrasion, a scar or a burn, reactions to medicaments, or phototoxic eruptions, and also subsequent to inflammatory diseases such as acne, eczema, psoriasis, lichen planus, lupus erythematosus, atopic dermatitis or cutaneous lymphoma.

PIHP is more common in dark phototypes, such as non-caucasian skin, in particular Asian, black or mixed-race skin.

The depigmenting agents or bleaching agents currently administered in the form of topical compositions make it possible to reduce the density of melanin in the epidermis. These agents are generally absorbed through the lower layers of the epidermis and slow down the formation of melanin.

Hydroquinone is a depigmenting agent, as are derivatives thereof such as benzyloxyphenol and hydroquinone monobenzyl ether. However, these agents have several drawbacks: hydroquinone is unstable in an alkaline medium and is oxidized in the form of quinine, which gives a brownish color to the compositions containing it; hydroquinone is irritant; it can also induce hypersensitivity reactions and, in certain rare cases, ochronosis; hydroquinone is also suspected of being carcinogenic; hydroquinone monobenzyl ether is not correctly metabolized when it is absorbed through the skin, and causes irreversible depigmentations. Methoxyphenol, a hydroquinone ether, is also known, but has the drawback of being relatively water-insoluble and difficult to incorporate into cosmetic or dermatological formulations.

A depigmenting composition comprising hydroquinone, retinoic acid and dexamethasone has been described (U.S. Pat. No. 3,856,934), but this composition is also irritant and can cause itching in the most extreme cases.

Various products of the vitamin C, fruit acid and sunscreen type have been developed for treating these pigmentation problems, but most contain unstable mixtures and are not very active.

Thus, need exists to treat hyperpigmentation spots and to eliminate skin defects generally due to the deposit of excess amounts of melanin.

Moreover, there exists a need to be able to bleach normally pigmented skin to increase the radiance of the complexion, or to make the appearance of the skin uniform.

The reasons which prompt bleaching of the skin may be diverse. Clear lightening of the complexion is often sought in sub-Saharan Africa and in Asian countries, with traditional or chemical solutions which generally have considerable harmful side effects on the appearance and the structure of the skin.

The paleness or the whiteness of the Asian face is obtained with molecules, such as arbutin, kojic acid or ascorbic acid, which may be poorly tolerated or irritant.

Therefore, need also exists for compositions which have a depigmenting or lightening activity and which are well-tolerated by the skin, in particular for non-caucasian skin, such as Hispanic, Indian, black, Asian or mixed-race skin.

Rucinol, or lucinol, or alternatively 4-butylresorcinol, is marketed as an agent for lightening brown spots related to pigmentation disorders.

### SUMMARY OF THE INVENTION

The present invention features compositions which provide improved effectiveness in the cosmetic and/or dermatological treatment of pigmentation, without the drawbacks of the prior art compositions.

A first aspect of the invention is a composition, preferably a dermatological or cosmetic composition, which comprises, in a physiologically acceptable medium, (i) at least one compound selected from among rucinol and salts thereof, and (ii) at least one retinoid (hereinafter referred to as “active agents (i) and (ii)”).

The term “dermatological composition” means a composition useful for treating and/or preventing pigmentary skin disorders.

The term “cosmetic composition” means a composition useful for making the skin more attractive and/or improving the physical appearance thereof.

The term “physiologically acceptable medium” means a medium compatible with the skin, the mucous membranes and/or the skin appendages.

A second aspect of the invention is the formulation of at least one compound selected from among rucinol and salts thereof, and of at least one retinoid, into a medicament useful in the treatment and/or prevention of pigmentation disorders. This invention also features the administration of a composition comprising, in a physiologically acceptable medium, at least one compound selected from among rucinol and salts thereof, and at least one retinoid, for the treatment and/or prevention of pigmentation disorders.

A third aspect of the invention is the administration of a cosmetic composition comprising, in a physiologically acceptable medium, at least one compound selected from among rucinol and salts thereof, and also comprising a retinoid, for the prevention and/or cosmetic treatment of skin pigmentation.

This invention also features a method for dermatological or cosmetic treatment of skin pigmentation, comprising the administration of a composition according to the invention, whether regime or regimen, to an individual in need of such treatment.

### DETAILED DESCRIPTION OF BEST MODE AND SPECIFIC/PREFERRED EMBODIMENTS OF THE INVENTION

Rucinol, or lucinol, or alternatively 4-butylresorcinol, is a compound of formula:

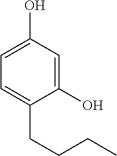

which is known and commercially available as an agent for lightening brown spots related to pigmentation conditions (product Iklen®).

The term “rucinol salts” means, in particular, salts formed with a pharmaceutically acceptable base, in particular an inorganic base such as sodium hydroxide, potassium hydroxide or aqueous ammonia, or an organic base such as lysine, arginine or N-methylglucamine, but also the salts formed with fatty amines such as dioctylamine, aminomethylpropanol and stearylamine.

Preferably, rucinol will be administered.

Surprisingly, a significant improvement in the depigmenting activity of rucinol has been demonstrated when it is in combination with a retinoid and, in particular, a depigmentation which is more rapid than the prior art compositions and than rucinol alone. In particular, it has now been discovered that an effective amount of retinoid acts in synergy with an effective amount of rucinol for the skin-depigmenting effect, and makes it possible to significantly reduce the side effects such as irritations.

Retinoids find various applications in dermatology. However, it is known that these compounds have considerable unwanted side effects, whether they are administered systemically or topically. They in particular cause strong irritations. These drawbacks of retinoids reflect that the advantageous effect of the compositions according to the invention could not be expected.

The retinoids that are useful in the context of the invention comprise in particular all-trans retinoic acid or tretinoin, 13-cis-retinoic acid or isotretinoin, acitretin, arotinoic acid, retinol, adapalene, tazarotene, retinaldehyde, etretinate, and the compounds described in WO 2006/066978, such as 3″-tert-butyl-4′-(2-hydroxyethoxy)-4″-pyrrolidin-1-yl[1,1′;3′,1″]terphenyl-4-carboxylic acid, the compounds of WO 2007/066041, including 2-hydroxy-4-[3-hydroxy-3-(5,6,7,8-tetrahydro-5,5,8,8-tetramethyl-2-naphthyl)-1-propynyl]benzoic acid or an enantiomer thereof, the compounds of WO 2005/056510, including 4′-(4-isopropylaminobutoxy)-3′-(5,5,8,8-tetramethyl-5,6,7,8-tetrahydronaphthalen-2-yl)biphenyl-4-carboxylic acid, the compounds of WO 2005/056510 including 4-{3-hydroxy-3-[4-(2-ethoxyethoxy)-5,5,8,8-tetramethyl-5,6,7,8-tetrahydronaphthalen-2-yl]prop-1-ynyl}benzoic acid, and the compounds of WO 2005/037772 including 4-[2-(3-tert-butyl-4-diethylaminophenyl)-2-hydroxyiminoethoxy]-2-hydroxybenzoic acid. Retinol and adapalene are particularly preferred.

The dermatological and cosmetic compositions according to the invention or the active agents (i) and (ii) thus make it possible to reduce skin pigmentation, and in particular local hyperpigmentations of the skin. In particular, when they are applied topically, they produce depigmentation of the area of skin to which they are applied.

The term “depigmentation” means obtaining a bleaching of an area of pigmented skin. When this area of skin is hyperpigmented, the term “depigmentation” means a bleaching of said area until a color similar to that of the neighboring skin is obtained.

The compositions of the invention or the active agents (i) and (ii) are particularly suitable for the treatment and/or prevention of pigmentation disorders such as:

melasma or chloasma,

lentigines, senile lentigo,

freckles or ephelides,

actinic keratosis,

planar pigmented seborrhoeic warts,

postinflammatory hyperpigmentations, in particular due to infections, allergic reactions, damage caused by trauma (such as an abrasion, a scar or a burn), reactions to medicaments, phototoxic eruptions, and also following inflammatory skin diseases (such as acne, eczema, psoriasis, rosacea, lichen planus, lupus erythematosus, atopic dermatitis and cutaneous lymphoma);

naevi,

genetically determined hyperpigmentations,

hyperpigmentations of metabolic origin.

The compositions according to the invention or the active agents (i) and (ii) are also useful in the cosmetics field, in particular for protection against the harmful aspects of sunlight, for preventing and/or combating photoinduced or chronological aging of the skin and of the skin appendages.

This invention also features a method of nontherapeutic cosmetic treatment for making the skin more attractive and/or for improving the surface appearance thereof, comprising the application, to the skin and/or its appendages, of a composition comprising at least one compound selected from rucinol and salts thereof, and at least one retinoid.

The compositions according to the invention advantageously comprise from 0.0001% to 20% by weight of at least one retinoid and from 0.0001% to 20% by weight of at least one compound selected from rucinol and salts thereof, relative to the total weight of the composition. Preferably, they comprise from 0.001% to 10% of at least one retinoid and from 0.01% to 15% of rucinol or salts thereof, and more particularly from 0.1% to 5% by weight of at least one retinoid and from 0.1% to 5% by weight of at least one compound selected from rucinol and salts thereof, relative to the total weight of the composition.

The compositions of the invention may also comprise any additive, normally administered in the pharmaceutical, dermatological or cosmetics field, which is compatible with rucinol or salts thereof and a retinoid.

Particularly exemplary thereof are sequestering agents, antioxidants, sunscreens, preservatives, for example DL-alpha-tocopherol, fillers, electrolytes, humectants, colorants, or customary inorganic or organic bases or acids, fragrances, essential oils, cosmetic active agents, moisturizers, vitamins, essential fatty acids, sphingolipids, agents for soothing and protecting the skin, such as allantoin, propenetrating agents, gelling agents, or a mixture thereof.

Of course, one skilled in the art will take care to select this or these optional additional compound(s), and/or the amount thereof, in such a way that the advantageous properties of the compositions according to the invention are not, or are not substantially, impaired.

These additives may be present in the composition in a proportion of from 0 to 20% by weight relative to the total weight of the composition.

Examples of sequestering agents are ethylenediaminetetraacetic acid (EDTA), and also derivatives thereof or salts thereof, dihydroxyethylglycine, citric acid and tartaric acid, or mixtures thereof.

Examples of preservatives are benzalkonium chloride, phenoxyethanol, benzyl alcohol, diazolidinylurea and parabens, or mixtures thereof.

Examples of humectants are glycerol and sorbitol.

The compositions of the invention can contain one or more propenetrating agents in preferential concentrations ranging from 0% to 20%, and more preferentially ranging from 0.6% to 3% by weight, relative to the total weight of the composition. Among the propenetrating agents, preferred are compounds such as propylene glycol, dipropylene glycol, propylene glycol dipelargonate, lauroglycol and ethoxydiglycol.

Advantageously, the compositions according to the invention may also contain one or more surfactants in preferential concentrations ranging from 0% to 10%, and more preferentially ranging from 0.1% to 2%, by weight, of the total weight.

Administration of the subject compositions may be carried out topically, enterally or orally, parenterally or ocularly.

Among these routes of administration, topical administration is particularly preferred. The term “topical administration” means application to the skin and/or the mucous membranes.

The compositions of the present invention may be in any of the galenical forms normally employed for topical application, in particular in liquid, pasty or solid form, and more particularly in the form of salves, aqueous, aqueous-alcoholic or oily solutions, dispersions of the lotion type, aqueous, anhydrous or lipophilic gels, powders, impregnated pads, syndets, wipes, sprays, patches, foams, sticks, shampoos, compresses, washing bases, emulsions with a liquid or semi-liquid consistency of the milk type, obtained by dispersion of a fatty phase in an aqueous phase (oil-in-water) or conversely (water-in-oil), or suspensions or emulsions of soft, semi-liquid or solid consistency of the cream, gel or ointment type. The composition may also be in the form of suspensions of microspheres or nanospheres or of lipid or polymeric vesicles or of polymeric or gelled patches for controlled release. This composition for topical application may be in anhydrous form, in aqueous form or in the form of an emulsion or else of microemulsions, microcapsules, microparticles or vesicular dispersions of ionic and/or non-ionic type.

Advantageously, the composition is in the form of an ointment, a cream, a lotion or a gel.

The aqueous phase of a composition according to the invention in the form of an emulsion may comprise water, a floral water such as cornflower water or a natural spring or mineral water, for example selected from water from Vittel, waters from the Vichy basin, water from Uriage, water from la Roche Posay, water from Bourboule, water from Enghien-les-Bains, water from Saint Gervais-les-Bains, water from Néris-les-Bains, water from Allevard-les-Bains, water from Digne, water from Maizieres, water from Neyrac-les-Bains, water from Lons-le-Saunier, water from Eaux-Bonnes, water from Rochefort, water from Saint Christau, water from Les Fumades and water from Tercis-les-bains, water from Avéve or water from Aix-les-Bains.

Said aqueous phase may be present at a content of from 10% to 90% by weight, relative to the total weight of the composition, preferably from 20% to 80% by weight.

The compositions according to the invention may contain a gelling agent at preferential concentrations ranging from 0.1% to 15%, and more preferentially ranging from 0.5% to 5%.

Exemplary thereof are gelling agents of the polyacrylamide family, such as the sodium acryloyldimethyltaurate copolymer/isohexadecane/polysorbate 80 mixture marketed under the trademark Simulgel™ 600 by Seppic™, the polyacrylamide/isoparaffin C13-14/laureth-7 mixture, for instance the product marketed under the trademark Sepigel 305™ by Seppic™, the family of acrylic polymers coupled to hydrophobic chains, such as the PEG-150/decyl/SMDI copolymer marketed under the trademark Aculyn 44™ (polycondensate comprising at least, as elements, a polyethylene glycol comprising 150 or 180 mol of ethylene oxide, decyl alcohol and methylenebis(4-cyclohexylisocyanate) (SMDI), at 35% by weight in a mixture of propylene glycol (39%) and water (26%)), the family of modified starches, such as the modified potato starch marketed under the trademark Structure Solanace™, or else mixtures thereof.

In order to further illustrate the present invention and the advantages thereof, the following specific examples are given, it being understood that same are intended only as illustrative and in nowise imitative. In said examples to follow, all parts and percentages are given by weight, unless otherwise indicated.

Examples of Formulations:

In this example, various specific formulations for a composition according to the invention have been illustrated.

Oral Administration:

(a) 0.2 g Tablet:

| | |
|---|---|
| Rucinol | 0.001 g |
| Adapalene | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

(b) Oral Suspension in 5 ml Vials:

| Rucinol | 0.001 | g |
|---|---|---|
| Retinol | 0.001 | g |
| Glycerol | 0.500 | g |
| 70% sorbitol | 0.500 | g |
| Sodium saccharinate | 0.010 | g |
| Methyl para-hydroxybenzoate | 0.040 | g |
| Flavoring | qs | |
| Purified water | qs 5 | ml |

B—Parenteral Administration:

| | | |
|---|---|---|
| Rucinol | 0.002 | g |
| Adapalene | 0.001 | g |
| Ethyl oleate | qs 10 | g |

C—Topical Administration:

(a) Salve:

| | |
|---|---|
| Rucinol | 0.010 g |
| Adapalene | 0.010 g |
| Isopropyl myristate | 81.700 g |
| Fluid liquid petroleum jelly | 9.100 g |
| Silica (“Aérosil 200” marketed by Degussa) | 9.180 g |

(b) Salve:

| | | |
|---|---|---|
| Rucinol | 0.300 | g |
| Retinol | 0.100 | g |
| Codex white petroleum jelly | qs 100 | g |

(c) Non-Ionic Water-in-Oil Cream:

| | | |
|---|---|---|
| Rucinol | 0.100 | g |
| Adapalene | 0.100 | g |
| Mixture of emulsive lanolin alcohols, | 39.900 | g |
| of waxes and of oils (“anhydrous eucerin” | | |
| marketed by BDF) | | |
| Methyl para-hydroxybenzoate | 0.075 | g |
| Propyl para-hydroxybenzoate | 0.075 | g |
| Sterile demineralized water | qs 100 | g |

(d) Lotion:

| | |
|---|---|
| Rucinol | 0.050 g |
| Tazarotene | 0.050 g |
| Polyethylene glycol (PEG 400) | 69.900 g |
| 95% ethanol | 30.000 g |

(e) Hydrophobic salve:

| | | |
|---|---|---|
| Rucinol | 0.250 | g |
| Adapalene | 0.150 | g |
| Isopropyl myristate | 36.400 | g |
| Silicone oil (“Rhodorsil 47 V 300” marketed | 36.400 | g |
| by Rhone-Poulenc) | | |
| Beeswax | 13.600 | g |
| Silicone oil (“Abil 300 000 cst” marketed | qs 100 | g |
| by Goldschmidt | | |

(f) Non-Ionic Oil-in-Water Cream:

| | | |
|---|---|---|
| Rucinol | 1.000 | g |
| Retinol | 1.000 | g |
| Cetyl alcohol | 4.000 | g |
| Glyceryl monostearate | 2.500 | g |
| PEG 50 stearate | 2.500 | g |
| Shea butter | 9.200 | g |
| Propylene glycol | 2.000 | g |
| Methyl para-hydroxybenzoate | 0.075 | g |
| Propyl para-hydroxybenzoate | 0.075 | g |
| Sterile demineralized water | qs 100 | g |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method of treating a pigmentation disorder, the method comprising administering to a subject in need of such treatment, for such period of time as required to elicit a desired effect, an effective amount of at least one compound selected from among rucinol and salts thereof and at least one retinoid.

2. The method as defined by claim 1, wherein said pigmentation disorder is selected from the group consisting of:
melasma,
lentigines,
senile lentigo,
freckles,
actinic keratosis,
a planar pigmented seborrhoeic wart,
postinflammatory hyperpigmentations,
allergic reactions,
traumas (abrasion, scar, burn), a reaction to a medicament, a phototoxic eruption, an inflammatory skin disease;
naevi,
a genetically determined hyperpigmentation, and
a hyperpigmentation of metabolic origin.

3. A method of combating photoinduced or chronological aging of the skin and of the skin appendages, the method comprising administering to a subject in need of such treatment, for such period of time as required to elicit a desired effect, an effective amount of at least one compound selected from among rucinol and salts thereof and at least one retinoid.

4. The method as defined by claim 1, wherein the at least one retinoid is selected from the group consisting of tretinoin, isotretinoin, acitretin, arotinoic acid, retinol, adapalene, tazarotene, retinaldehyde, etretinate, 3″-tert-butyl-4′-(2-hydroxyethoxy)-4″-pyrrolidin-1-yl[1,1′;3′,1″]terphenyl-4-carboxylic acid, 2-hydroxy-4-[3-hydroxy-3-(5,6,7,8-tetrahydro-5,5,8,8-tetramethyl-2-naphthyl)-1-propynyl]benzoic acid or an enantiomer thereof, 4′-(4-isopropylaminobutoxy)-3′-(5,5,8,8-tetramethyl-5,6,7,8-tetrahydronaphthalen-2-yl)biphenyl-4-carboxylic acid, 4-{3-hydroxy-3-[4-(2-ethoxyethoxy)-5,5,8,8-tetramethyl-5,6,7,8-tetrahydronaphthalen-2-yl]prop-1-ynyl}benzoic acid and 4-[2-(3-tert-butyl-4-diethylaminophenyl)-2-hydroxyiminoethoxy]-2-hydroxybenzoic acid.

5. The method as defined by claim 1, wherein the at least one retinoid comprises adapalene.

6. The method as defined by claim 1, wherein the at least one retinoid comprises retinol.

7. The method as defined by claim 1, wherein the at least one compound is delivered in a composition comprising from 0.0001% to 20% by weight of the at least one compound selected from among rucinol and salts thereof, and from 0.0001% to 20% by weight of at least one retinoid.

8. The method as defined by claim 7, wherein the composition comprises from 0.01% to 15% by weight of rucinol and salts thereof, and from 0.001% to 10% by weight of at least one retnoid.

9. The method as defined by claim 7, wherein the composition comprises from 0.1% to 5% by weight of at least one compound selected from rucinol and salts thereof, and from 0.1% to 5% by weight of at least one retnoid.

10. The method as defined by claim 7, wherein the composition is formulated for topical administration.

11. The method as defined by claim 10, wherein the composition is in a form selected from the group consisting of a salve, an aqueous solution, a lotion, a gel, a powder, an impregnated pad, a syndet, a wipe, a spray, a patch, a foam, a stick, a shampoo, a compress, a washing base, an emulsion, a cream, an ointment, a suspension of microsphere or nanosphere, a lipid vesicle, and a polymeric vesicle.

12. The method as defined by claim 3, wherein the at least one retinoid is selected from the group consisting of tretinoin, isotretinoin, acitretin, arotinoic acid, retinol, adapalene, tazarotene, retinaldehyde, etretinate, 3″-tert-butyl-4′-(2-hydroxyethoxy)-4″-pyrrolidin-1-yl[1,1′;3′,1″]terphenyl-4-carboxylic acid, 2-hydroxy-4-[3-hydroxy-3-(5,6,7,8-tetrahydro-5,5,8,8-tetramethyl-2-naphthyl)-1-propynyl]benzoic acid or an enantiomer thereof, 4′-(4-isopropylaminobutoxy)-3′-(5,5,8,8-tetramethyl-5,6,7,8-tetrahydronaphthalen-2-yl)biphenyl-4-carboxylic acid, 4-{3-hydroxy-3-[4-(2-ethoxyethoxy)-5,5,8,8-tetramethyl-5,6,7,8-tetrahydronaphthalen-2-yl]prop-1-ynyl}benzoic acid and 4-[2-(3-tert-butyl-4-diethylaminophenyl)-2-hydroxyiminoethoxy]-2-hydroxybenzoic acid.

13. The method as defined by claim 3, wherein the at least one retinoid comprises adapalene.

14. The method as defined by claim 3, wherein the at least one retinoid comprises retinol.

15. The method as defined by claim 3, wherein the at least one compound is delivered in a composition comprising from 0.0001% to 20% by weight of the at least one compound selected from among rucinol and salts thereof, and from 0.0001% to 20% by weight of at least one retinoid.

16. The method as defined by claim 15, wherein the composition comprises from 0.01% to 15% by weight of rucinol and salts thereof, and from 0.001% to 10% by weight of at least one retnoid.

17. The method as defined by claim 15, wherein the composition comprises from 0.1% to 5% by weight of at least one compound selected from rucinol and salts thereof, and from 0.1% to 5% by weight of at least one retnoid.

18. The method as defined by claim 15, wherein the composition is formulated for topical administration.

19. The method as defined by claim 18, wherein the composition is in a form selected from the group consisting of a salve, an aqueous solution, a lotion, a gel, a powder, an impregnated pad, a syndet, a wipe, a spray, a patch, a foam, a stick, a shampoo, a compress, a washing base, an emulsion, a cream, an ointment, a suspension of microsphere or nanosphere, a lipid vesicle, and a polymeric vesicle.
